SEQUENCE LISTING (34 sequences)

SEQ ID NO: 1 - DNA, 20 bp, Artificial Sequence
  Synthetic
ggacatctaagggcatcaca

SEQ ID NO: 2 - DNA, 20 bp, Artificial Sequence
  Synthetic
tcaagaacgaaagtcggagg

SEQ ID NO: 3 - DNA, 21 bp, Artificial Sequence
  Synthetic
catgtacgttgctatccaggc

SEQ ID NO: 4 - DNA, 21 bp, Artificial Sequence
  Synthetic
ctccttaatgtcacgcacgat

SEQ ID NO: 5 - DNA, 23 bp, Artificial Sequence
  Synthetic
acaactttggtatcgtggaagga

SEQ ID NO: 6 - DNA, 19 bp, Artificial Sequence
  Synthetic
gccatcacgccacagtttc

SEQ ID NO: 7 - DNA, 22 bp, Artificial Sequence
  Synthetic
gaggtgacccttgtctctgtgt

SEQ ID NO: 8 - DNA, 19 bp, Artificial Sequence
  Synthetic
agcccagaggcctgtgcca

SEQ ID NO: 9 - DNA, 27 bp, Artificial Sequence
  Synthetic
ccagatcactgggcagcatgtggcacc

SEQ ID NO: 10 - DNA, 27 bp, Artificial Sequence
  Synthetic
agcagggtctagagcagagcagctgcc

SEQ ID NO: 11 - DNA, 20 bp, Artificial Sequence
  Synthetic
actgacgtgcctctccctcc

SEQ ID NO: 12 - DNA, 21 bp, Artificial Sequence
  Synthetic
ccgtatctcccttccctgatt

SEQ ID NO: 13 - DNA, 22 bp, Artificial Sequence
  Synthetic
atctgtccctcacagcagggtc

SEQ ID NO: 14 - DNA, 19 bp, Artificial Sequence
  Synthetic
ggctgacctaaagccacct

SEQ ID NO: 15 - DNA, 23 bp, Artificial Sequence
  Synthetic
aattaggtccagagtgagttaac

SEQ ID NO: 16 - DNA, 24 bp, Artificial Sequence
  Synthetic
acttgcatgtcagaggatataatg

SEQ ID NO: 17 - DNA, 25 bp, Artificial Sequence
  Synthetic
catcaagaaacagtaaccagtaatg

SEQ ID NO: 18 - DNA, 23 bp, Artificial Sequence
  Synthetic
aaggcctcagctgtttggctaag

SEQ ID NO: 19 - DNA, 23 bp, Artificial Sequence
  Synthetic
ttgactggaagtgtcgcatcacc

SEQ ID NO: 20 - DNA, 24 bp, Artificial Sequence
  Synthetic
catgtgacagaacacagtgacatg

SEQ ID NO: 21 - DNA, 20 bp, Artificial Sequence
  Synthetic
gtgaagtcctcccagcccgc

SEQ ID NO: 22 - DNA, 21 bp, Artificial Sequence
  Synthetic
ctcccatcagaactgccgacc

SEQ ID NO: 23 - DNA, 25 bp, Artificial Sequence
  Synthetic
tggaggacaagtaatgatctcctgg

SEQ ID NO: 24 - DNA, 24 bp, Artificial Sequence
  Synthetic
aagagagaccagagcccagacctg

SEQ ID NO: 25 - DNA, 23 bp, Artificial Sequence
  Synthetic
gccatggctgtggtttgtgatgg

SEQ ID NO: 26 - DNA, 24 bp, Artificial Sequence
  Synthetic
atcctagccccttgtggacatagg

SEQ ID NO: 27 - DNA, 21 bp, Artificial Sequence
  Synthetic
ggactcttgctgggcatgtgg

SEQ ID NO: 28 - DNA, 21 bp, Artificial Sequence
  Synthetic
ccactcagagttctcccatgg

SEQ ID NO: 29 - DNA, 21 bp, Artificial Sequence
  Synthetic
ccatgggagaactctgagtgg

SEQ ID NO: 30 - DNA, 21 bp, Artificial Sequence
  Synthetic
tcccttcacatgctgaggtgg

SEQ ID NO: 31 - DNA, 22 bp, Artificial Sequence
  Synthetic
agactcctgagcagaacctctg

SEQ ID NO: 32 - DNA, 20 bp, Artificial Sequence
  Synthetic
agccagcacagctcagccac

SEQ ID NO: 33 - DNA, 22 bp, Artificial Sequence
  Synthetic
actgtctagaccagactggagg

SEQ ID NO: 34 - DNA, 21 bp, Artificial Sequence
  Synthetic
gagggtgctcttagccacagg

(19) United States
(12) Patent Application Publication
(10) Pub. No.: US 2012/0308567 A1
(43) Pub. Date: Dec. 6, 2012

(54) USE OF TYROSINE KINASE INHIBITORS FOR TREATMENT OF PROLACTINOMA

(75) Inventors: Hidenori Fukuoka, Culver City, CA (US); Odelia Cooper, Los Angeles, CA (US); Shlomo Melmed, Los Angeles, CA (US)

(73) Assignee: CEDARS-SINAI MEDICAL CENTER, Los Angeles, CA (US)

(21) Appl. No.: 13/576,564

(22) PCT Filed: Jan. 31, 2011
(86) PCT No.: PCT/US11/23237

§ 371 Date: Aug. 1, 2012

(30) Foreign Application Priority Data
Feb. 1, 2010 (US) 61300367
Sep. 30, 2010 (US) 61388490

(51) Int. Cl. A61K 31/517; A61K 39/395; C12Q 1/48; A61K 31/713; A61K 31/5377; A61K 38/16; A61K 31/7088

(52) U.S. Cl. 424/135.1; 514/19.2; 424/172.1; 514/44 R; 514/266.24; 424/133.1; 514/44 A; 514/234.5; 435/15

(57) ABSTRACT

The invention relates to methods and kits for the treatment of prevention of and lowering the chances of developing prolactinomas by the administration of a tyrosine kinase inhibitor, such as lapatinib.

This application claims priority to U.S. Ser. No. 61/388,490 filed Sep. 30, 2010 and to U.S. Ser. No. 61/300,367 filed Feb. 1, 2010, the contents of all of which are herein incorporated by reference.

This invention was made with government support under Grant Nos. CA07597 and K23DK085148-01 awarded by the National Institutes of Health. The government has certain rights in the invention.

### FIELD OF INVENTION

This invention relates to treatment of prolactinoma with tyrosine kinase inhibitors.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Lapatinib (or lapatinib ditosylate) (marketed in the United States as Tykerb® by GlaxoSmithKline) inhibits the tyrosine kinase activity associated with EGFR (epidermal growth factor receptor) and HER2/neu (Human EGFR type 2). Lapatinib is a protein kinase inhibitor that inhibits receptor signal processes by binding to the ATP-binding pocket of the EGFR/HER2 protein kinase domain, preventing self-phosphorylation and subsequent activation of the signal mechanism.

Pituitary tumors are detected in up to 25% of random autopsies (1), and prolactinomas constitute the most prevalent hormone-secreting pituitary adenomas (2, 3). Prolactinomas are benign monoclonal adenomas which hypersecrete PRL, and usually present with amenorrhea, galactorrhea, and infertility in females, and sexual dysfunction, and sellar mass effects including headaches, visual dysfunction, and/or hypopituitarism, in males (4). Both sexes are at increased risk of osteoporosis (5). Drug treatment for this commonly encountered tumor is limited to dopamine agonists. If dopamine agonists are not effective in normalizing PRL levels or in shrinking tumor size, or if the patient cannot tolerate medication side effects, trans-sphenoidal adenoma resection may be considered (6, 7). However surgical cure rates for patients with invasive macroprolactinomas are poor, and even if resected, large prolactinomas tend to recur post-operatively (5). Thus, there is currently a need in the art for treatments for prolactinoma.

As PRL gene expression is regulated by the ErbB family receptor ligands, EGF and heregulin (HRG) (8-10), the inventors believed that EGF receptor (EGFR) inhibition would be effective for control of PRL secretion and tumor load in prolactinomas (8). HER2/ErbB2 is an orphan receptor which amplifies signaling by ErbB-containing heterodimers including the EGFR, by enhancing ligand binding affinity and/or receptor recycling and stability (11-13). HER2/ErbB2 gene amplification or overexpression is associated with poor clinical outcomes in breast and non-small cell lung cancers (14-16), and transgenic mice overexpressing HER2/ErbB2 develop mammary tumors and lung metastases (17, 18). HER2/ErbB2 receptors are expressed in pituitary tumors (19-21), including prolactinomas (9). However, HER2/ErbB2 receptor function in pituitary tumors remains unknown. Lapatinib, a small-molecule tyrosine kinase inhibitor (TKI), targets both EGFR/ErbB 1 and HER2/ErbB2, and reversibly binds cytoplasmic receptor kinase ATP-binding sites abrogating both MAPK and Akt pathway signaling (22).

As shown herein, the inventors demonstrated functional in vitro and in vivo roles of HER2/ErbB2 in rat prolactinoma hormone regulation and cell proliferation, and also show effects on HER2/ErbB2 overexpressing rat prolactinoma cells, and on primary human prolactinoma cells derived from surgically resected prolactinoma tissue. The results support a rationale for ErbB targeted therapy in patients harboring PRL-secreting pituitary adenomas.

### SUMMARY OF THE INVENTION

The invention provides methods for treating prolactinoma in a subject. The methods comprise providing a composition comprising a tyrosine kinase inhibitor and administering to the subject an effective amount of the composition, thereby treating prolactinoma in a subject.

The invention further provides methods for inhibiting and/or reducing prolactinoma in a subject. The methods comprise providing a composition comprising a tyrosine kinase inhibitor and administering to the subject an effective amount of the composition, thereby inhibiting and/or reducing prolactinoma in a subject.

Methods for promoting prolactinoma prophylaxis are also provided herein. The methods comprise providing a composition comprising a tyrosine kinase inhibitor and administering to the subject an effective amount of the composition, thereby promoting prolactinoma prophylaxis in a subject.

The invention also provides methods for screening for compounds that inhibit tyrosine kinase. The screening method comprises contacting the compound of interest with a cell expressing tyrosine kinase and assaying for amounts of prolactin (PRL). A reduction in the amount of prolactin compared to the control is indicative of the compound of interest inhibiting tyrosine kinase.

The invention further provides kits for treatment of prolactinoma, inhibition of prolcatinoma, reduction of prolactinoma and/or promotion of prolactinoma prophylaxis in a subject. The kit comprises a composition comprising a tyrosine kinase inhibitor and instructions for use of the composition for treatment of prolactinoma, inhibition of prolcatinoma, reduction of prolactinoma and/or promotion of prolactinoma prophylaxis in a subject.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows that HER2 enhances PRL mRNA expression and secretion in accordance with various embodiments of the present invention. A. GH3 cells stably expressing HER2CA or pcDNA3 (empty vector) were treated with 5 nM EGF or 6 nM HRG for 10 min, and Western blotting performed. B. GH3 cells stably expressing HER2CA or pcDNA3 were treated with 5 nM EGF or 6 nM HRG for the indicated times, and Realtime PCR analysis performed, and C. GH and PRL secretion in the culture medium was determined by RIA.

Hormone secretion levels were normalized for cell numbers. Values are mean±SEM. *p<0.05, ** p<0.01. Representative results are from triplicate samples in at least two or more independent experiments.

FIG. 2 shows that HER2 enhances cell proliferation in accordance with various embodiments of the present invention. A. GH3 cells stably expressing HER2CA or pcDNA3 were plated (20,000 per well) in 12 well plates for the indicated times, and cells counted. B. GH3 cells stably expressing HER2CA or pcDNA3 were plated (4,000 per well) in 96 well plates for the indicated times, and WST assay performed. C. Stably transfected GH3 cells were seeded (4,000 per well) for colony-forming assays, and EGF added to the upper layer with cells, and to the soft agar surface with serum-depleted media every third day. Colonies were counted from 5 randomly selected fields. Values are mean±SEM. *p<0.05, ** p<0.01, ^{‡}p<0.05 vs pcDNA3 control, ^{†}p<0.05 vs HER2CA control, ^{‡‡}p<0.05 vs pcDNA3 control, ^{††}p<0.05 vs HER2CA control. Representative results are from triplicate samples in at least two or more independent experiments.

FIG. 3 shows that lapatinib attenuates HER2 signaling and suppresses PRL more than gefitinib in accordance with various embodiments of the present invention. A. GH3 cells stably expressing HER2CA were pretreated with gefitinib or lapatinib (0.1-10 μM) for 45 min prior to induction with EGF (5 nM) for 10 min, and Western blotting performed. B. GH3 cells stably expressing HER2CA were treated with gefitinib or lapatinib (1

μM) for the indicated times, and Realtime PCR analysis performed. C. GH and PRL secretion in the culture medium determined by RIA. Hormone secretion levels were normalized for cell numbers, Values are mean±SEM. *p<0.05, **p<0.01. Representative results are from triplicate samples in at least two or more independent experiments.

FIG. 4 shows dose dependent effects of gefitinib or lapatinib on HER2CA GH3 proliferation and apoptosis in accordance with various embodiments of the present invention. A. GH3 cells stably expressing HER2CA were treated with gefitinib or lapatinib (0.1-10 μM) for 24 hr, and cells counted. B. Stably transduced GH3 cells were seeded (4,000 per well) for colony-forming assay, and gefitinib or lapatinib (0.1-10 μM) added with serum-depleted media every third day. Colonies were counted from 5 randomly selected fields. C. GH3 cells stably expressing HER2CA were treated with gefitinib or lapatinib (0.1-10 μM) for 24 hr, and Western blotting performed. The ratio of cleaved caspase-3 vs GAPDH was calculated by densitometric analysis of each treatment group. Values are mean±SEM. *p<0.05, **p<0.01, ^{††}p<0.01 vs same dose gefitinib treatment. Representative results are from triplicate samples in at least two or more independent experiments.

FIG. 5 shows that lapatinib attenuates HER2CA GH3 tumor growth and hormone secretion in vivo more than gefitinib in accordance with various embodiments of the present invention. A. GH3 cells stably expressing HER2CA (3×10⁶ cells/rat, 0.2 ml with matrigel) or pcDNA3 were inoculated subcutaneously in WF rats (4-5 weeks of age). Tumor volumes were measured 8 days after cell inoculation. **p<0.01 vs. pcDNA3. B-C. Three days after inoculation, rats were divided into three groups; vehicle (0.5% methylcellose, and 0.5% tween80/PBS), gefitinib (100 mg/kg), and lapatinib (100 mg/kg). (B). Tumor weights were measured after euthanasia (C). Serum PRL levels were measured by RIA (D), and ex vivo Realtime PCR performed (E). Tumor volumes were calculated using the formula, π/6×large diameter×small diameter². Values are mean±SEM. *p<0.05, **p<0.01 vs. vehicle group.

FIG. 6 shows that lapatinib attenuates estrogen induced pituitary lactotroph tumor growth and hormone secretion in vivo in accordance with various embodiments of the present invention. 17β-estradiol-filled capsules or empty capsule were inoculated subcutaneously in F344 rats (4-5 weeks of age) for one month. A. Western blotting analysis was performed using collected pituitary or pituitary tumor. Next 17β-estradiol-filled capsules were inoculated subcutaneously in F344 rats (4-5 weeks of age) for 2 months. After capsule excision, oral vehicle or lapatinib was administered for 2 weeks. B. Representative pituitary tumor induced by estrogen was attenuated by lapatinib treatment. C. Tumor weights were measured after euthanasia. D. Serum PRL and GH levels were measured by RIA. Values are mean±SEM. *p<0.05 vs. vehicle group.

FIG. 7 shows that lapatinib attenuates PRL secretion and mRNA expression in human prolactinoma cell cultures in accordance with various embodiments of the present invention. A, B, D, and E. After trans-sphenoidal surgery of human prolactinomas, tumor cells were cultured. Prolactinoma cells (Tumor A) were treated with lapatinib (0.1-10 μM) or gefitinib (10 μM) for 24 hr, and Realtime PCR of PRL performed (A). PRL levels in culture media were measured using RIA (B). H&E and PRL staining of tumor and confocal immunocytochemistry of EGFR and HER2 (Tumor A) (C), or for Tumor B (F). Magnification of these figures was 100×. Prolactinoma cells (Tumor B) were treated with lapatinib (0.01-10 μM) or gefitinib (0.01-10 μM) for 24 hr, and Realtime PCR performed (D). PRL levels in culture media were measured using RIA (E). Prolactinoma cells (Tumor B) were treated with U0126 (0.1-5 μM) for 24 hr, and Realtime PCR of PRL performed (G). Values are mean±SEM. *p<0.05, **p<0.01 vs. control. ***p<0.001 vs. control.

### DETAILED DESCRIPTION OF THE INVENTION

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Conditions” and “disease conditions,” as used herein may include, but are in no way limited to any form of prolactinoma.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Tyrosine kinase inhibitors,” as used herein, refer to molecules and pharmaceuticals, the administration of which to a subject result in the inhibition of tyrosine kinase—an enzyme that can transfer a phosphate group from ATP to a tyrosine residue in a protein. Examples of tyrosine kinase inhibitors include, but are not limited to, gefitinib, erlotinib hydrochloride (marketed as Tarceva® by Genentech and OSI Pharmaceuticals), lapatinib or lapatinib ditosylate (marketed as Tykerb® by GlaxoSmithKline), neratinib, trastuzumab, and pharmaceutical salts, equivalents and analogs thereof.

### Therapeutic Methods of the Invention

The present invention is based, at least in part, on these findings and thus, present invention describes methods and kits for using tyrosine kinase inhibitors to treat conditions in a subject, such as prolactinomas. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. While not wishing to be bound by any particular theory, the inventors believe that Her2/Neu potently induces PRL secretion and regulates experimental prolactinoma cell proliferation, and thus, as Her2/Neu pituitary signaling is abrogated by tyrosine kinase inhibitors, this receptor is an effective target for medical therapy of prolactinomas.

The present invention provides for a method of treating prolactinoma in a subject in need thereof, comprising providing a quantity of a tyrosine kinase inhibitor and administering the quantity of the tyrosine kinase inhibitor to the subject to treat the prolactinoma. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. In various embodiments of the invention, the subject has HER2-overexpressing pituitary adenomas, such as a HER2-overexpressing prolactinoma.

The present invention also provides for a method of inhibiting prolactinoma in a subject in need thereof, comprising providing a quantity of a tyrosine kinase inhibitor and administering the quantity of the tyrosine kinase inhibitor to the subject to inhibit prolactinoma. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. In various embodiments of the invention, the subject has HER2-overexpressing pituitary adenomas, such as a HER2-overexpressing prolactinoma.

The present invention provides for a method of lowering a subject's chances of developing prolactinoma in a subject in need thereof, comprising providing a quantity of a tyrosine kinase inhibitor and administering the quantity of the tyrosine kinase inhibitor to the subject to lower the subject's chances of developing prolactinoma. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. In various embodiments of the invention, the subject has HER2-overexpressing pituitary adenomas, such as a HER2-overexpressing prolactinoma.

The invention further provides methods for reducing prolactinoma tumor size in a subject in need thereof, comprising: providing a quantity of a tyrosine kinase inhibitor and administering the quantity of the tyrosine kinase inhibitor to the subject to reduce prolactinoma tumor size. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. In various embodiments of the invention, the subject has HER2-overexpressing pituitary adenomas, such as a HER2-overexpressing prolactinoma.

The invention also provides methods for promoting prolactinoma prophylaxis in a subject in need thereof, comprising providing a quantity of a tyrosine kinase inhibitor and administering the quantity of the tyrosine kinase inhibitor to the subject to promote prolactinoma prophylaxis. In an embodiment, the tyrosine kinase inhibitor is lapatinib or gefitinib. In the preferred embodiment of the invention, the tyrosine kinase inhibitor is lapatinib, a salt thereof, or a pharmaceutical equivalent thereof. In various embodiments of the invention, the subject has HER2-overexpressing pituitary adenomas, such as a HER2-overexpressing prolactinoma.

In other embodiments of the invention, the tyrosine kinase inhibitor is any one or more of a small molecule, a peptide, an antibody or a fragment thereof, a nucleic acid molecule, or a combination thereof. In an embodiment of the invention, the tyrosine kinase inhibitor is a small molecule. In one embodiment, the small molecule is lapatinib, a salt thereof, or a pharmaceutical equivalent thereof. In another embodiment, the small molecule is gefitinib, a salt thereof, or a pharmaceutical equivalent thereof. In a further embodiment, the tyrosine kinase inhibitor is a nucleic acid molecule, wherein the nucleic acid molecule inhibits tyrosine kinase. For example, the nucleic acid molecule that inhibits tyrosine kinase may be an siRNA molecule of tyrosine kinase.

In a further embodiment of the invention, the tyrosine kinase inhibitor is an anti-tyrosine kinase antibody. In an embodiment, the antibody specifically binds tyrosine kinase so as to inhibit tyrosine kinase. The antibody may be any one or more of a monoclonal antibody or fragment thereof, a polyclonal antibody or a fragment thereof, a chimeric antibody, a humanized antibody, a human antibody or a single chain antibody. These antibodies can be from any source, e.g., rat, dog, cat, pig, horse, mouse or human. Fragments of antibodies may be any one or more of Fab, F(ab′)₂, Fv fragments or fusion proteins.

The subjects treated by the present invention include mammalian subjects, including, human, monkey, ape, dog, cat, cow, horse, goat, pig, rabbit, mouse and rat.

Various methods may be utilized to administer the composition of the claimed methods, including but not limited to aerosol, nasal, oral, transmucosal, transdermal, parenteral, implantable pump, continuous infusion, topical application, capsules and/or injections.

### Dosages of the Invention

In some embodiments of the invention, the effective amounts of tyrosine kinase inhibitor in the composition can be in the range of about 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day. In one embodiment of the invention, the tyrosine kinase inhibitor is lapatinib. In another embodiment of the invention, the tyrosine kinase inhibitor is gefitinib.

In further embodiments of the invention, the effective amount of tyrosine kinase inhibitor for use with the claimed methods may be in the range of 100-200 mg/kg, 200-300 mg/kg, 300-400 mg/kgy, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg or 2900-3000 mg/kg. In one embodiment of the invention, the tyrosine kinase inhibitor is lapatinib. In another embodiment of the invention, the tyrosine kinase inhibitor is gefitinib

Typical dosages of an effective amount of a tyrosine kinase inhibitor, such as lapatinib, can be in the ranges recommended by the manufacturer where known therapeutic compounds are used, and also as indicated to the skilled artisan by the in vitro responses or responses in animal models. For example, lapatinib is currently recommended at 1,250 mg (5 tablets) given orally once daily on days 1-21 continuously in combination with capecitabine 2,000 mg/m²/day (administered orally in two doses approximately twelve hours apart) on days 1-14 in a repeating 21-day cycle. Lapatinib should be taken at least one hour before or one hour after a meal. The same or similar dosing can be used in accordance with various embodiments of the present invention, or an alternate dosage may be used in connection with alternate embodiments of the invention, with or without capecitabine. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

### Screening Methods of the Invention

Another aspect of the invention relates to assays and methods for identifying compounds that inhibit tyrosine kinase. In one embodiment, the method comprises contacting tyrosine kinase in a tyrosine kinase positive cell with the compound of interest and subsequently determining whether the contact results in altered amounts of prolactin. In an embodiment of the claimed methods, an alteration in the amount of prolactin is a decrease in the amount of prolactin. In one embodiment, a decrease in the amount of prolactin secretion is indicative that the molecule of interest is an inhibitor of tyrosine kinase. In another embodiment, decrease in the amount of prolactin synthesized is indicative that the molecule of interest is an inhibitor of tyrosine kinase. In a further embodiment, decrease in the amount of nucleic acid (for example, mRNA) encoding prolactin is indicative that the molecule of interest is an inhibitor of tyrosine kinase.

The compound of interest that inhibits tyrosine kinase may be any one or more of a small molecule, a peptide, an antibody or a fragment thereof and a nucleic acid molecule.

Assays that may be employed to indentify compounds that inhibit tyrosine kinase include but are not limited to microarray assay, quantitative PCR, Northern blot assay, Southern blot assay, Western blot assay immunohistochemical assays, binding assays, gel retardation assays or assys using yeast two-hybrid systems. A person skilled in the art can readily employ numerous techniques known in the art to determine whether a particular agent inhibits tyrosine kinase.

### Pharmaceutical Compositions

In various embodiments, the present invention provides pharmaceutical compositions including a pharmaceutically acceptable excipient along with a therapeutically effective amount of a tyrosine kinase inhibitor, such as lapatinib. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Kits of the Invention

The present invention is also directed to kits to treating prolactinomas. The kit is an assemblage of materials or components, including at least one of the inventive compositions.

Thus, in some embodiments the kit contains a composition including a tyrosine kinase inhibitor, such as lapatinib, as described above.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or prevent prolactinomas in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing a tyrosine kinase inhibitor, such as lapatinib. The packaging material generally has an external label which indicates the contents and/or purpose of the kit and/or its components.

### EXAMPLES

The following example is provided to better illustrate the claimed invention and is not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Experimental Methods

DMEM/F12 (phenol red free), and penicillin/streptomycin were purchased from Invitrogen. EGF was from Sigma, and NRG1-β1/HRG1-β1 was from R&D systems. Lapatinib (Tykarb) was from LC Laboratories and gefitinib (Iressa) was purchased from Biaffin GmbH & Co. U0126 was purchased from Promega.

### Stable Transfected Cells

GH3 rat lacto-somatotroph tumor cells secreting PRL and GH were purchased from the American Type Culture Collection. HER2CA cells were generated by transfection with pcDNA3-HER2CA (V654E) purchased from Addgene (Addgene plasmid 16259). Stable colonies were selected in the presence of 500 μg/ml G418 (Invitrogen). A vector control cell line pcDNA3 was simultaneously established by transfecting pcDNA3 that lacked inserted cDNA. After selection and propagation of stable transfectants, cells were cultured in DMEM/F12 medium containing 15% horse serum, 2.5% FBS, penicillin/streptomycin, and 500 μg/m G418. After synchronization by serum starvation (medium containing 0.2% bovine serum albumin for ˜24 h), cells with treatment agents was grown in fresh serum-depleted medium (0.2% bovine serum albumin), and samples collected at the indicated times.

### Quantitative PCR

Total RNA was extracted with Trizol reagent (Invitrogen) according to instructions of the manufacturer. The amount and the integrity of RNA were assessed by measurement of absorbance at 260 and 280 nm. Total RNA was reverse-transcribed into first-strand cDNA using iScript cDNA synthesis kit (Bio-Rad Laboratories, Inc.) according to the manufacturer. Quantitative PCR reactions were carried out in the iQ5 Multicolor Real-time PCR Detection System (Bio-Rad Laboratories, Inc.) as described (8). Certified RT² primer assays for rat GH, PRL and human PRL were purchased from SuperArray. Primer sequences (Invitrogen) were 5′-GGACATCTAAGGGCATCACA-3′ (18S rRNA forward (F)), 5′-TCAAGAACGAAAGTCGGAGG-3′ (18S rRNA reverse (R)), 5′-CATGTACGTTGCTATCCAGGC-3′ (human (3-actin F), 5′-CTCCTTAATGTCACGCACGAT-3′ (human (3-actin R), 5′-ACAACTTTGGTATCGTGGAAGGA-3′ (human GAPDH F), 5′-GCCATCACGCCACAGTTTC-3′ (human GAPDH R).

### Western Blotting

After completion of treatments, cells were placed on ice and washed with cold PBS. For protein extraction, cells were lysed in 100 μl RIPA buffer (Sigma) containing complete protease inhibitor cocktail tablets (Roche Molecular Biochemicals) and phosphatase inhibitor cocktail 2 (Sigma). Lysates were centrifuged at 13,000×g for 10 min at 4° C. and protein concentrations determined by BCA™ protein assay reagent (Thermo Scientific). Western blot analysis was performed according to the guidelines of NuPAGE electrophoresis system protocol (Invitrogen). In brief, whole cell lysates (˜50 μg protein per lane) were heated for 5 min at 100° C., respectively. Proteins were separated on 4 to 12% NuPAGE Bis-Tris gels and electro-transferred for 3 h to polyvinylidene difluoride membranes (Invitrogen). Membranes were blocked for 1 h in 5% nonfat dry milk or 5% bovine serum albumin in TBS-T buffer, and incubated overnight with primary antibody. The following primary antibodies were used: anti-pErk1/2, anti-Erk1/2, anti-Akt, anti-pEGFR (Tyr1068), anti-EGFR, anti-pHER3/ErbB3 (Tyr1289), and anti-Cleaved Caspase-3 from Cell Signaling Technology, anti-pNeu (Tyr1248), anti-Neu (C-18), anti-HER3/ErbB3, anti-PRL, and anti-GAPDH from Santa Cruz Biotechnology, and anti-pAkt (Ser473) from Abcam. After washing with TBS-T, membranes were incubated with peroxidase-conjugated secondary antibody for 1 h (5% nonfat dry milk or 5% bovine serum albumin in TBS-T buffer). Blots were washed and hybridization signals measured by enhanced chemiluminescence detection system (Amersham).

### Soft Agarose Colony-Forming Assay

Base layers consisting of growth medium containing 0.6% low-melting point agarose (Invitrogen) were poured onto 6-well plates and allowed to solidify. Cells (8×10³ per well) were plated in triplicate in top layers consisting of growth medium containing 0.3% agarose. Seven to ten days later, cells were stained with 0.2% iodonitrotetrazolium chloride (Invitrogen), and colonies composed of 50 cells were counted manually in 5 randomly selected fields.

### Cell Proliferation Assay

pcDNA3 and HER2CA cells were plated at a density of 2×10⁴ per well in 12-well plates or 4×10³ cells per well in 96-well plates with growth medium. For 12-well plates, cells were counted by hemocytometer at the indicated times. For 96-well plates, premixed WST-1 cell proliferation reagent (Roche Molecular Biochemicals) was added (1:10) at the indicated times and incubated for 4 h at 37° C. in a humidified atmosphere maintained at 5% CO2, and absorbance was then measured at 450 nm. WST-1 is a colorimetric assay for quantification of cell proliferation and cell viability, based on the cleavage of the tetrazolium salt by mitochondrial dehydrogenases in viable cells.

### Hormone Assay

RIAs for rat GH and PRL were performed in duplicate, using reagents provided by the National Hormone and Pituitary Program, National Institute of Diabetes and Digestive and Kidney Diseases (Harbor-UCLA Medical Center, Torrance, Calif., USA). Iodination of GH and PRL (5 μg) with iodine-125 (500 μCi) (PerkinElmer life & Analytical Sciences, Boston, Mass., USA) mixed with 0.1 mg Iodo-Gen (Pierce, Rockford, Ill.) was performed using 10-ml columns prepared by G-75 Sephadex (Sigma Chemical Co.). Low interspecies cross-reactivity of the GH and PRL assays were previously shown (8).

### Animals

In accordance with Institutional Animal Care and Use Committee guidelines, 4-5 wk old female Wistar-Furth rats (Harlan Sprague Dawley, Inc.) were inoculated with pcDNA3 (n=6) or HER2CA (n=6) cells (3×10⁶ cells/rat). Rats were fed with a commercial pelleted diet ad libitum and tap water. Tumor volumes were measured with a caliper and calculated using the formula, π/6×large diameter×small diameter² as previously described (8). Next, 4-5 wk female WF rats were inoculated s.c. with HER2CA cells and from day three after s.c. inoculation, rats were divided into three groups (n=10/group) and treated with gefitinib (100 mg/kg), lapatinib (100 mg/kg), or vehicle (0.5% methylcellose, 0.5% tween80/PBS; 100 μl) via oral gavage daily for 1 week. 500 μl blood was collected twice for hormone assessment (before inoculation, 2 days after inoculation) by retro-orbital bleeding. This procedure was performed under isoflurane inhalational anesthesia via a nose cone connected to a rodent anesthesia machine (Kent Scientific Corporation). On the last treatment day (day 7), rats were euthanized within 3 h of drug administration. Cardiac blood was collected with 18-gauge syringes and tumors excised and weighed. Fragments of each tumor were fixed in formalin and embedded in paraffin for immunohistochemical staining, preserved in RNA later solution (Ambion) for subsequent RNA extraction and frozen in liquid nitrogen for subsequent protein extraction.

Next, 4-5 wk ovariectomized female Fishcer-344 rats (Harlan Sprague Dawley, Inc.) were s.c. implanted with an 17β-estradiol-filled SILASTIC brand capsule (Dow Corning Corp., Medical Grade Tubing Special; length, 3 cm; od, 0.125 in.; id, 0.062 in.) under isoflurane inhalational anesthesia. Two months thereafter, when serum PRL levels reached 3000 ng/ml, 17β-estradiol-filled capsules were extracted, and animals were treated with lapatinib (100 mg/kg) or vehicle (0.5% methylcellose, 0.5% tween80/PBS; 100 μl) via oral gavage twice a day for 2 weeks. 500 μl blood was collected for hormone assessment by retro-orbital bleeding under isoflurane inhalational anesthesia. On the last treatment day (day 14), rats were euthanized within 3 h of drug administration. Cardiac blood was collected and pituitary tumors excised and weighed. Fragments of each tumor were fixed in formalin and embedded in paraffin, preserved in RNA later solution, or frozen in liquid nitrogen.

### Cultures of Prolactinoma-Derived Cells

Human prolactinoma tissues were obtained at the time of surgery (Pituitary Center, Cedars-Sinai Medical Center) and transferred in 0.3% BSA containing DMEM according to an IRB-approved protocol. After washing with medium, tumor tissues were chopped with a sterile scalpel into approximately 1-2 mm pieces. Tissues were rinsed and digested with DMEM containing 0.3% BSA, 0.35% collagenase, and 0.15% hyaluronidase at 37° C. for 30 min The mixture was centrifuged at 1,500 rpm for 5 min at 4° C., and the cell pellet resuspended in an appropriate volume of culture medium containing 10% FBS and antibiotics in 48 well plates. After 24 hr incubation with serum depleted starvation medium (DMEM with 0.3% BSA), treatment agents were added with fresh serum-depleted medium (0.3% BSA), and medium collected for RIA. RNA was extracted after 24 hr treatment. Medium was also collected at baseline. To normalize for cell number effect, the PRL value of treated medium was divided by that of pre-treatment starvation medium to obtain a treated value for each well (n=4).

### Immunofluorescence

Tumor specimens were fixed in 10% formalin and embedded in paraffin. After deparaffinization, and antigen retrieval, slides were blocked in 10% goat serum in 1% bovine serum albumin-PBS and then incubated overnight with primary antibody at 4° C. The following antibodies were used: rabbit polyclonal anti-EGFR (ab2430; 1:50; abcam), anti-Neu (C-18; 1:100; Santa Cruz Biotechnology). Following washes, slides were incubated with Alexa Fluor goat anti-rabbit 488 (H+L) secondary antibodies (1:500; Invitrogen) and with Topro3 (Invitrogen) for 2 h at room temperature, and following such, slides were mounted with Prolong Gold antifade reagent (Invitrogen). Confocal microscope images were obtained using TCS-SP confocal scanner (Leica Microsystems) in a dual-emission mode to separate autofluorescence from specific staining. A spectral window from 500-550 nm wavelength detected emission of Alexa 488. A second window from 560-620 nm detected the autofluorescence contribution to the signal. In the final images, Alexa 488 appears green. Autofluorescence appears red. The two images were merged, so that all autofluorescence appears yellow, and true signals appear green.

### DNA Extraction and Sequencing Analysis

Genomic DNA was isolated by QiAamp® DNA Micro Kit (QIAGEN) from frozen tissues following the manufacturer's instructions. EGFR and HER2 sequences of the seven exons of the TK domain (exons 18-24) were detected using PCR based direct sequencing. PCR amplification was done in 50 μl volume containing genomic DNA using Expand High Fidelity PCR System (Roche). DNA was amplified for 1 cycle at 94° C. for 2 minutes, 30 cycles at 94° C. for 20 seconds, 55-65° C. for 30 seconds, and 68° C. for 15 seconds, followed by 7 minutes extension at 68° C. Primer Sequences for EGFR were 5′-GAGGTGACCCTTGTCTCTGTGT-3′ (exon 18 F), 5′-AGCCCAGAGGCCTGTGCCA-3′ (exon 18 R), 5′-CCAGATCACTGGGCAGCATGTGGCACC-3′ (exon 19 F), 5′-AGCAGGGTCTAGAGCAGAGCAGCTGCC-3′ (exon 19 R), 5′-ACTGACGTGCCTCTCCCTCC-3′ (exon 20 F), 5′-CCGTATCTCCCTTCCCTGATT-3′ (exon 20 R), 5′-ATCTGTCCCTCACAGCAGGGTC-3′ (exon 21 F), 5′-GGCTGACCTAAAGCCACCT-3′ (exon 21 R), 5′-AATTAGGTCCAGAGTGAGTTAAC-3′ (exon 22 F), 5′ -ACTTGCAT GTCAGAGGATATAAT G-3′ (exon 22 R), 5′ -CAT CAAGAAACAGTAACCAGTAAT G-3′ (exon 23 F), 5′-AAGGCCTCAGCTGTTTGGCTAAG-3′ (exon 23 R), 5′ -TT GACT GGAAGT GTC GCATCACC-3′ (exon 24 F), 5′-CATGTGACAGAACACAGTGACATG-3′ (exon 24 R), and for HER2 were 5′-GTGAAGTCCTCCCAGCCCGC-3′ (exon 18 F), 5′-CTCCCATCAGAACTGCCGACC-3′ (exon 18 R), 5′-TGGAGGACAAGTAATGATCTCCTGG-3′ (exon 19 F), 5′-AAGAGAGACCAGAGCCCAGACCTG-3′ (exon 19 R), 5′-GCCATGGCTGTGGTTTGTGATGG-3′ (exon 20 F), 5′-ATCCTAGCCCCTTGTGGACATAGG-3′ (exon 20 R), 5′-GGACTCTTGCTGGGCATGTGG-3′ (exon 21 F), 5′-CCACTCAGAGTTCTCCCATGG-3′ (exon 21 R), 5′-CCATGGGAGAACTCTGAGTGG-3′ (exon 22 F), 5′-TCCCTTCACATGCTGAGGTGG-3′ (exon 22 R), 5′-AGACTCCTGAGCAGAACCTCTG-3′ (exon 23 F), 5′-AGCCAGCACAGCTCAGCCAC-3′ (exon 23 R), 5′-ACTGTCTAGACCAGACTGGAGG-3′ (exon 24 F), 5′-GAGGGTGCTCTTAGCCACAGG-3′ (exon 24 R). Sequencing was performed by Sequetech DNA Sequencing Service.

### Statistical Analysis

Results are expressed as mean±SEM. Differences were assessed by one-way ANOVA following by Scheffe's F test. P<0.05 was considered significant.

### Example 2

The inventors recently reported pathways underlying in vitro and in vivo regulation of pituitary tumor gene expression and cell proliferation by EGF, heregulin and ErbB receptor ligand signaling. As Her2/Neu, an ErbB receptor family member, is overexpressed in prolactinomas, the inventors tested the role of Her2/Neu in prolactinoma hormone regulation and cell proliferation to support the rationale for targeting this receptor for drug therapy of those tumors.

The inventors generated constitutively active Her2/Neu stable GH3 cell transfectants (Her2CA-GH3), and tested PRL gene expression, and cell proliferation. They inoculated hormone-secreting Her2CA-GH3 cells to WF rats, and treated them with oral lapatinib, a dual tyrosine kinase inhibitor of Herl/EGFR and Her2/Neu, or gefitinib, a tyrosine kinase inhibitor of Herl/EGFR. They also treated primary cultured pituitary cells derived from human prolactinomas with lapatinib.

After selection and propagation, MAPK phosphorylation, and PRL mRNA levels were markedly enhanced (˜250-fold) in Her2CA-GH3 compared to empty vector stable transfectants (EV-GH3). PRL secretion was induced 100-fold in Her2CA-GH3 cells (p<0.01), and stable transfectants exhibited increased cell proliferation (1.8 fold, p<0.01). Her2CA-6113 cells also showed higher colony formation in soft agar (31±1.6 vs 12±1.3 control colonies per field, p<0.01). Lapatinib blocked Herl/EGFR and Her2/Neu signaling molecules, and suppressed PRL expression>gefitinib (˜50% suppression with gefitinib, p<0.05; ˜70% suppression with lapatinib, p<0.01). Lapatinib suppressed colony formation in soft agar (˜80%, p<0.01) more than gefitinib (˜50%, p<0.01) and induced cleaved-caspase 3, a marker of apoptosis. Tumors in rats implanted sc with Her2CA-GH3 were larger than those implanted with EV-GH3 (766±53 vs 568±38 mm3, p<0.05). Her2CA-GH3 tumor transfectants implanted in rats decreased in size (−40%, p<0.05) after one week of lapatinib treatment. Next the inventors treated human primary prolactinoma tumor cell cultures with lapatinib or gefitinib. Both human PRL mRNA expression and PRL secretion were respectively suppressed by lapatinib (˜80%, p<0.01, and ˜70%, p<0.01).

The inventors found that Her2/Neu potently induces PRL secretion and regulates experimental prolactinoma cell proliferation.

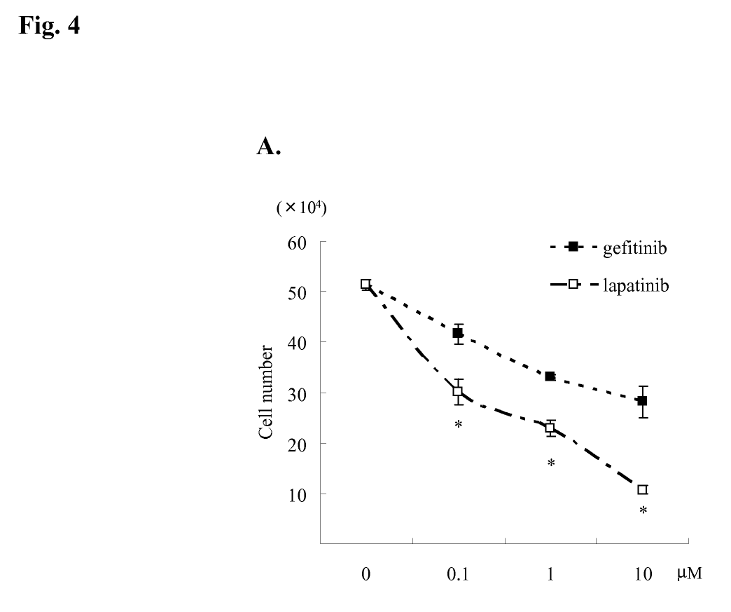

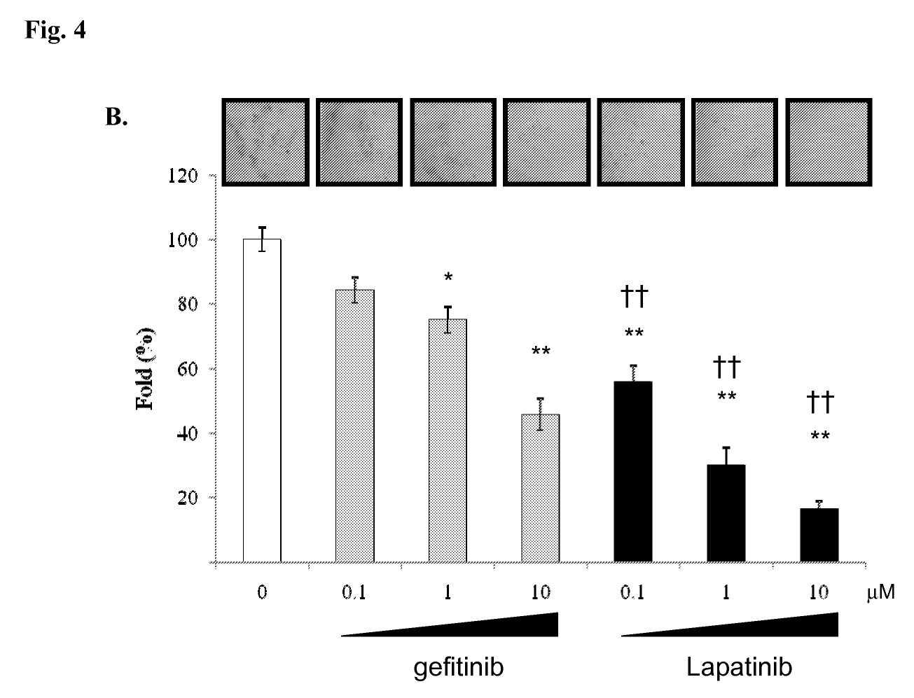

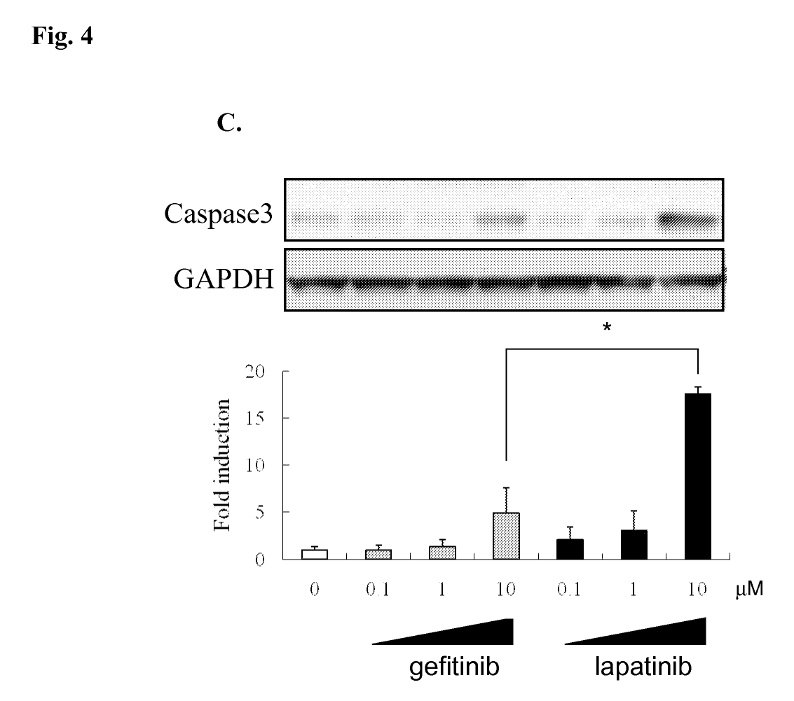

HER2CA colonies were observed with lapatinib than gefitinib treatment (FIG. 4B). As shown in FIG. 4C, lapatinib>gefitinib dose-dependently induced cleaved caspase3, a pro-apoptotic mechanism for the observed inhibitory effects on cell growth.

### Example 3

### HER2/ErbB2 Overexpression Enhances PRL Expression and Secretion and Cell Proliferation

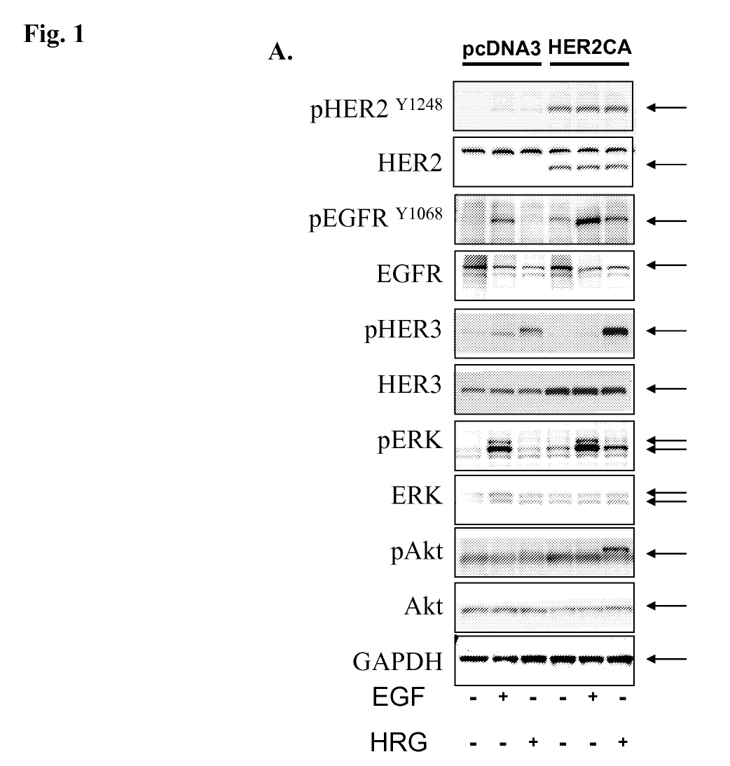

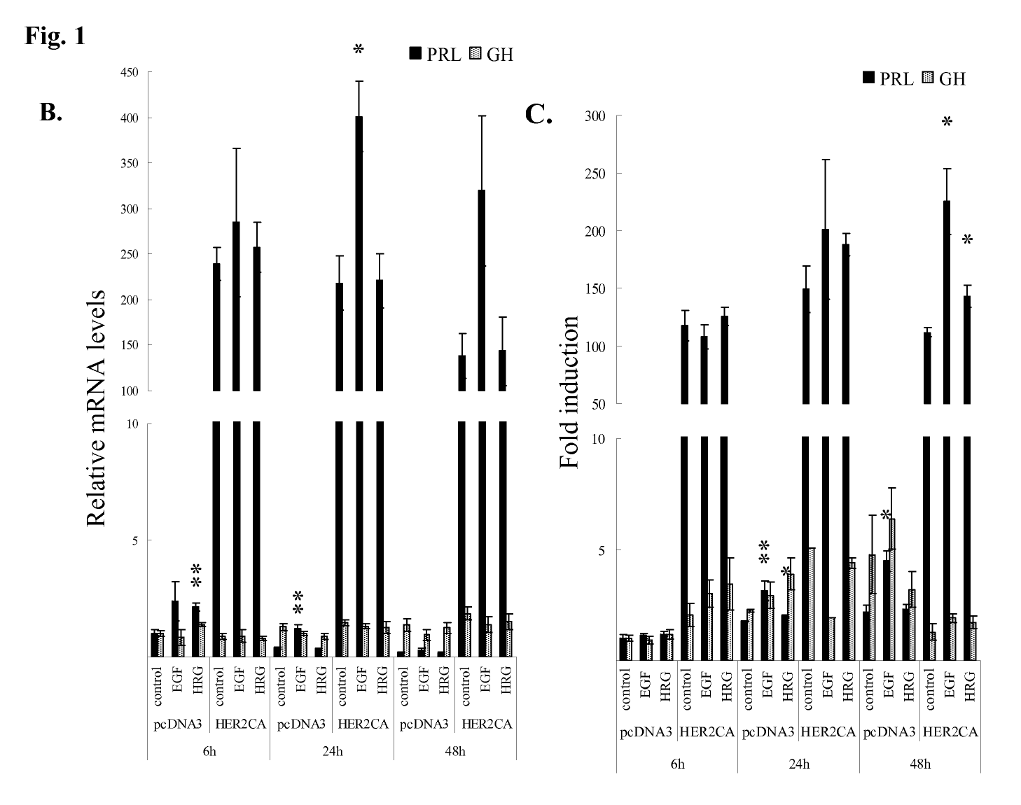

GH3 rat lactosomatotroph pituitary tumor cells (GH3) were stably transfected with an expression vector containing the constitutively active form (V654E) of HER2/ErbB2 cDNA (HER2CA) or empty vector (pcDNA3). Western blot results showed that HER2 and phosphor-HER2 protein were induced approximately 10-fold in HER2CA transfectants (FIG. 1A). Cells expressing HER2CA also contained higher levels of phosphorylated EGFR, MAPK, and Akt, but less phospho-HER3 than pcDNA3 transfectants (FIG. 1A). EGF induction of both phosphorylated EGFR and MAPK, and HRG induction of both phosphorylated HER3 and Akt, was also enhanced in HER2CA cells (FIG. 1A). HER2CA cells exhibited a marked and selective induction (˜250 fold) of PRL mRNA (P<0.0001), with no observed effects on GH mRNA expression (FIG. 1B). PRL, but not GH, secretion into the HER2CA cell medium was enhanced about 100-fold (FIG. 1C). As shown previously (8, 9), both EGF and HRG induced PRL expression and secretion approximately 2-fold in each respective transfectant (FIGS. 1B and C).

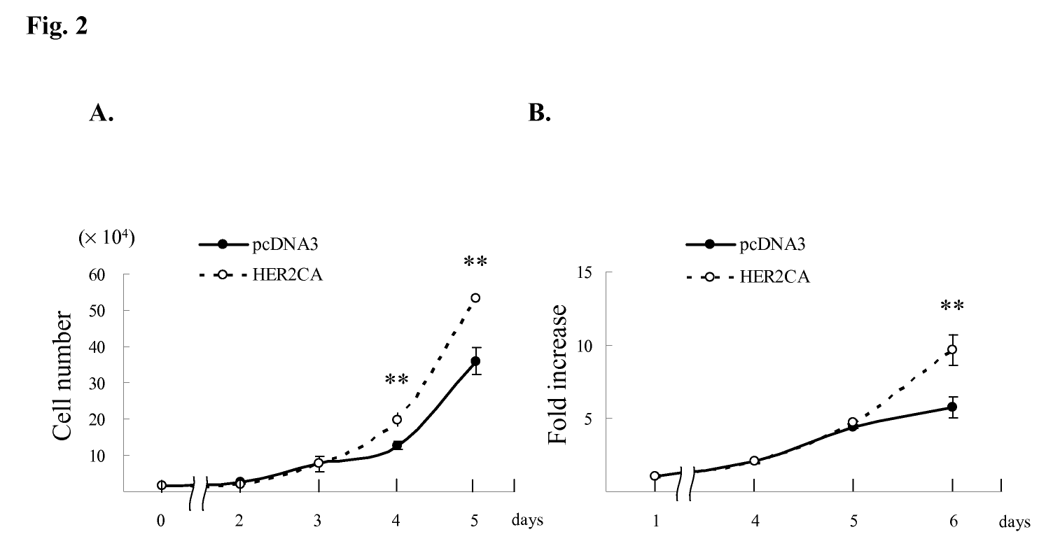

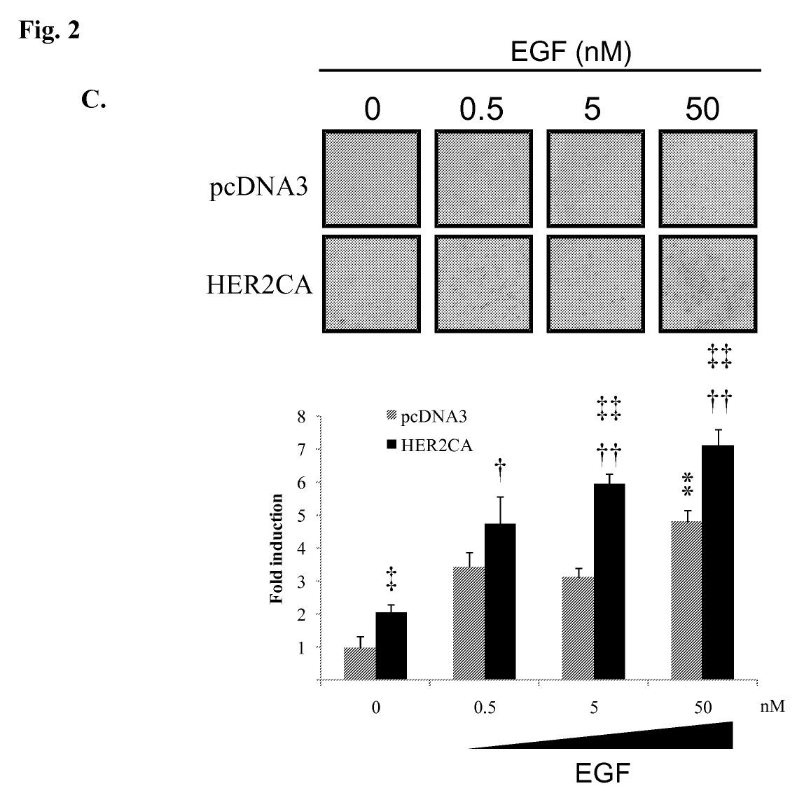

HER2CA cells proliferated faster than pcDNA3 transfectants as assessed by both water-soluble tetrazolium salt (WST-1) assays (1.8-fold on d 6; P<0.01), and by cell counts (1.5-fold on d 5; P<0.01) (FIGS. 2A and B). Furthermore, colony formation in soft agar was enhanced in HER2CA cells, and addition of EGF further enhanced dose-dependent colony formation 10 d after seeding both pcDNA3 (˜5-fold, P<0.01) and HER2CA transfectants (˜8 fold, P<0.01) (FIG. 2C).

### Example 4

### Lapatinib Suppresses PRL Expression and Secretion and Cell Growth

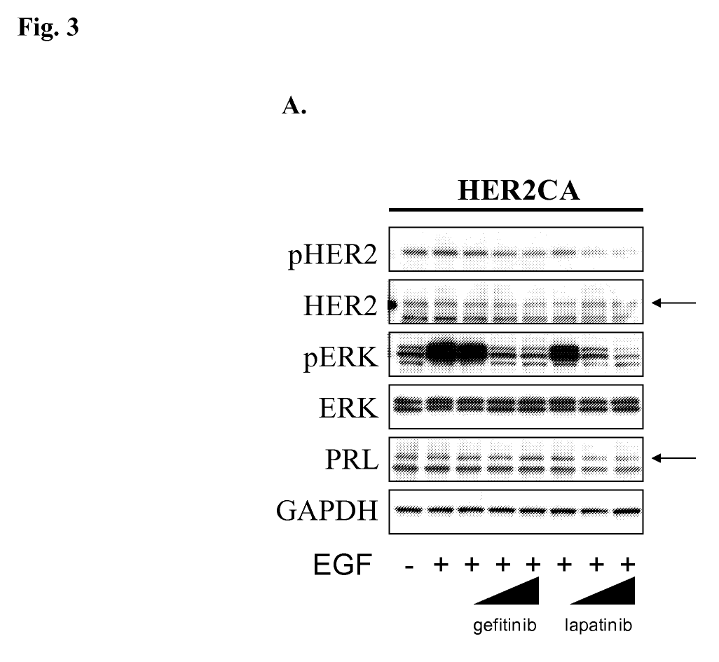

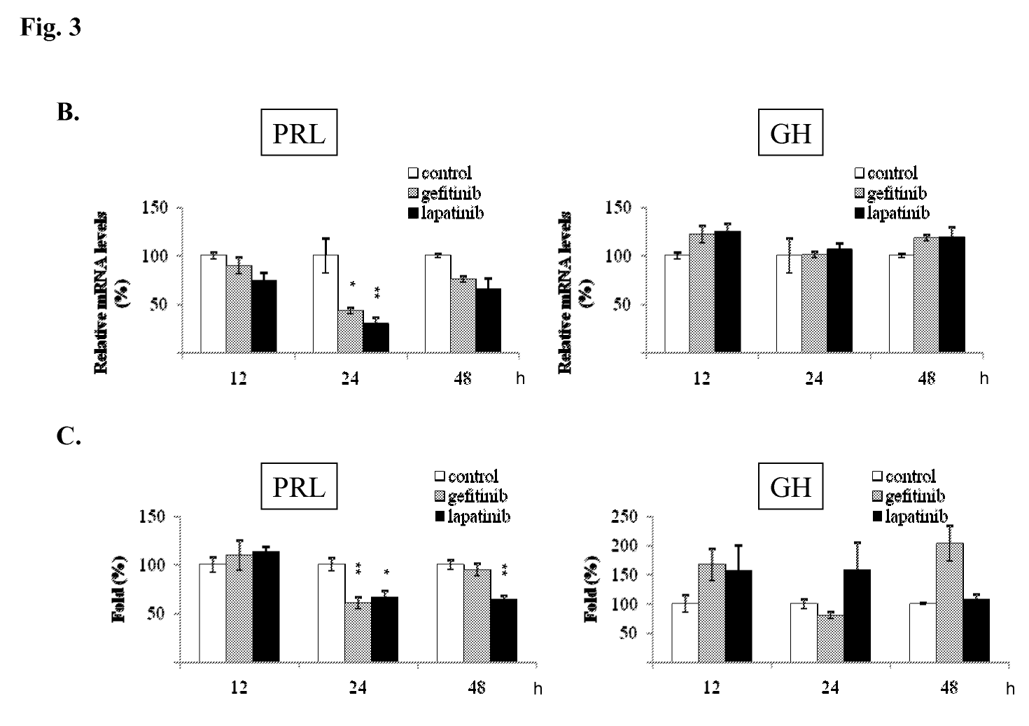

Because prolactinoma hormone secretion and cell proliferation were enhanced in constitutively active HER2/ErbB2 transfectants, the inventors tested effects of lapatinib, a dual TKI, for both EGFR/ErbB1 and HER2/ErbB2 and compared these with effects of gefitinib, an EGFR/ErbB1 TKI (23), Lapatinib attenuated EGF-induced HER2 and MAPK autophosphorylation more markedly than gefitinib, and intracellular PRL levels were decreased by lapatinib but not by gefitinib (FIG. 3A). PRL mRNA levels were decreased by both drugs, although lapatinib elicited more marked suppression (FIG. 3B). In contrast, GH mRNA levels were not altered by either drug. Lapatinib also specifically suppressed PRL secretion by 40% at 24 (P<0.05) and 48 h (P<0.01), whereas treatment with gefitinib suppressed PRL secretion only at 24 h (FIG. 3C). These results indicate that lapatinib is more effective than gefitinib in suppressing PRL synthesis and secretion in these PRL-secreting adenoma cells.

After 24 h treatment, cell number was decreased in a dose-dependent manner by lapatinib more than gefitinib (FIG. 4A). Colony formation in soft agar was also decreased in a dose dependent manner by the TKIs, and fewer HER2CA colonies were observed with lapatinib than gefitinib treatment, (FIG. 4B). As shown in FIG. 4C, lapatinib more than gefitinib dose-dependently induced cleaved caspase3, a pro-apoptotic mechanism for the observed inhibitory effects on cell growth.

### Example 5

### Lapatinib Action on HER2CA GH3 Tumors In Vivo

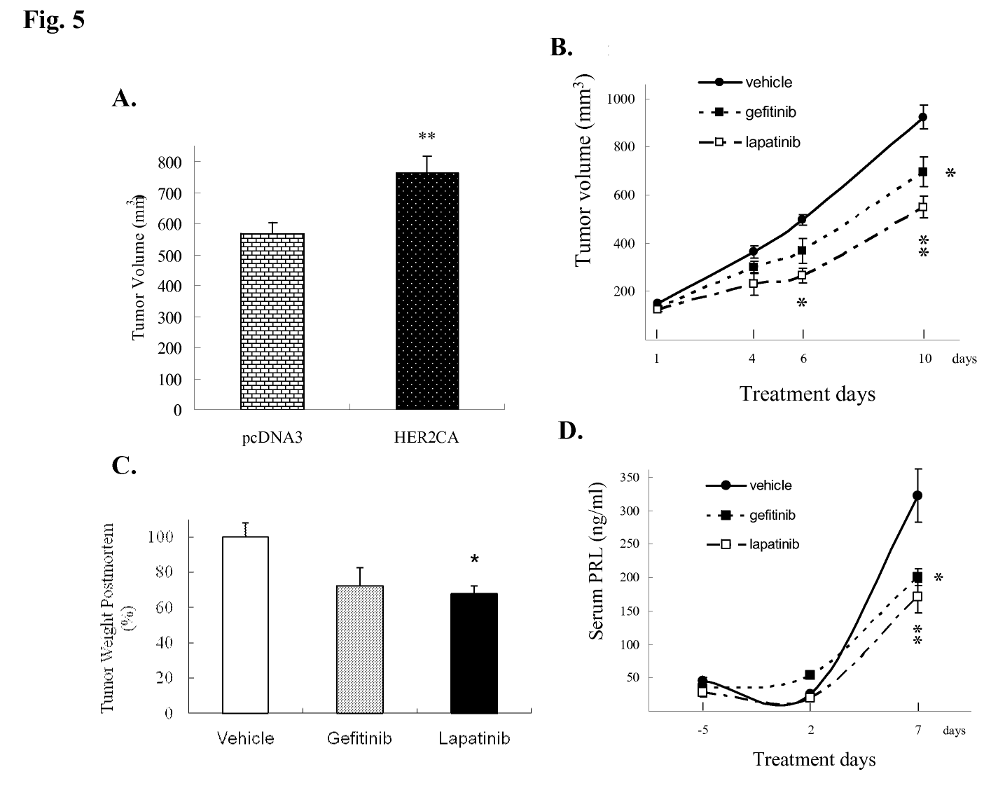

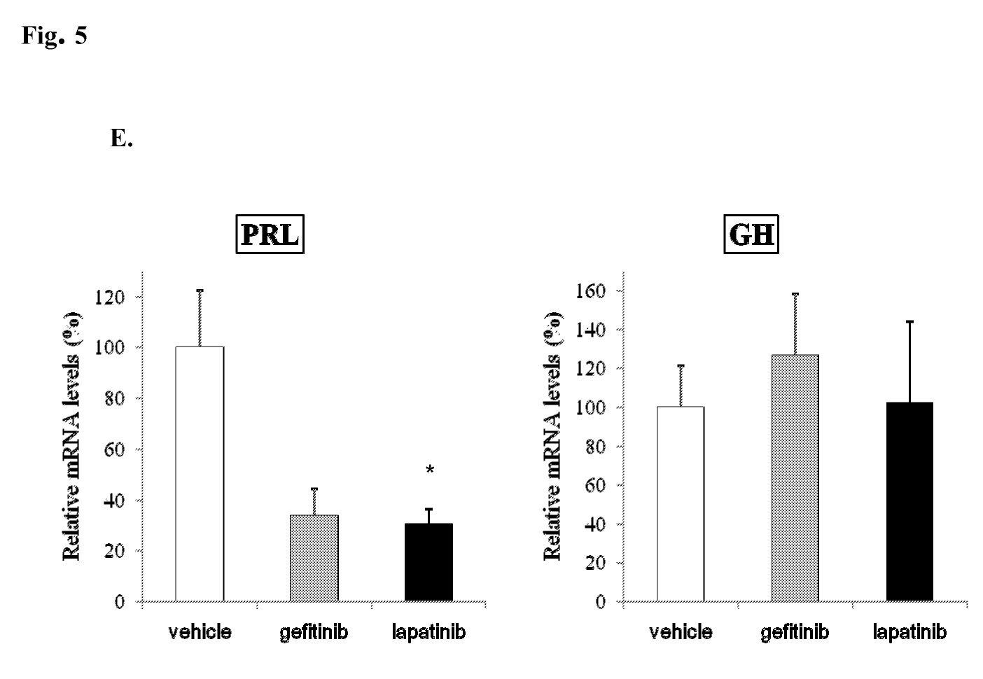

To evaluate in vivo effects of HER2 overexpression on lactosomatotroph tumor growth, HER2CA or pcDNA3 transfectants were inoculated subcutaneously into female Wistar-Furth rats. As shown in FIG. 5A, HER2CA tumors thus generated were larger than controls (766±53 vs 568±38 mm³, p<0.05). Next, the effects of lapatinib administration on tumor growth and hyperprolactinemia were examined. Three days after tumor cell inoculation, rats were randomly assigned to receive daily lapatinib (100 mg/kg body weight), gefitinib (100 mg/kg body weight), or vehicle (0.5% methylcellulose, 0.5% tween80/PBS; 100 μl) by oral gavage (n=10 rats per group) for 10 days. As shown in FIG. 5B, HER2CA tumor volume was attenuated by lapatinib>gefitinib (vehicle, 924±48 mm³; gefitinib, 695±63 mm³ p<0.05; and lapatinib, 549±45 mm³ p<0.01). After euthanasia, tumors were excised and immediately weighed and processed. Postmortem tumor weights were suppressed by lapatinib ˜40% (p=0.019), while gefitinib suppressed tumor mass ˜30% (p=0.052). Serum PRL levels were also attenuated by both drugs, with lapatinib treatment resulting in ˜50% suppression (p<0.01), and gefitinib treatment ˜40% suppression (p<0.05) (FIG. 5D). Tumor PRL mRNA levels were also attenuated by the treatments (˜70%, p<0.05), while tumor GH mRNA levels were unaltered (FIG. 5E).

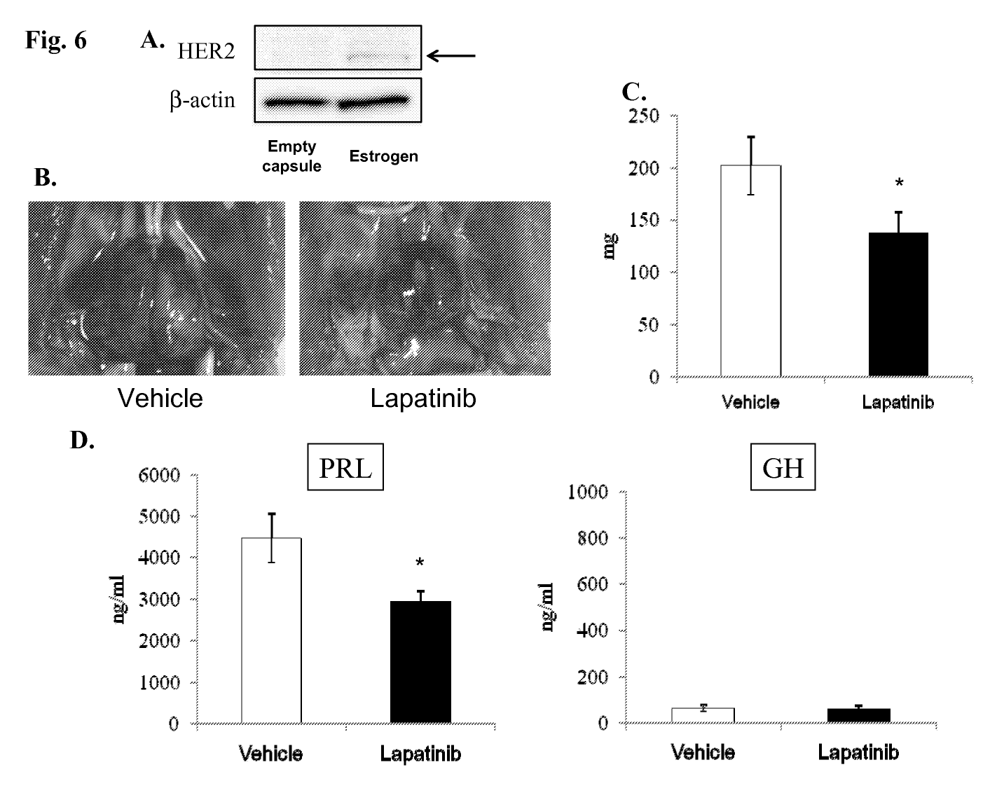

Next, the inventors tested Fischer-344 rats treated with 17β-estradiol as a model for pituitary prolactinomas (24). One month after inoculation of 17β-estradiol-filled capsules, HER2 expression was elevated in pituitary tumors (FIG. 6A). Two months after inoculation, serum PRL levels were elevated to 3330±185 ng/ml. The inventors then extracted the capsule, and initiated oral lapatinib or vehicle treatment for a subsequent 2 weeks. As shown in FIG. 6B, pituitary tumor growth induced by estrogen was attenuated by lapatinib. Postmortem tumor measurements showed that lapatinib suppressed tumor weight by ˜35% (p<0.05, FIG. 6C), and serum PRL levels by ˜35% (p<0.05), while serum GH levels were not altered (FIG. 6D).

### Example 6

### Lapatinib Attenuates PRL mRNA Expression and PRL Secretion in Human Prolactinoma Cells

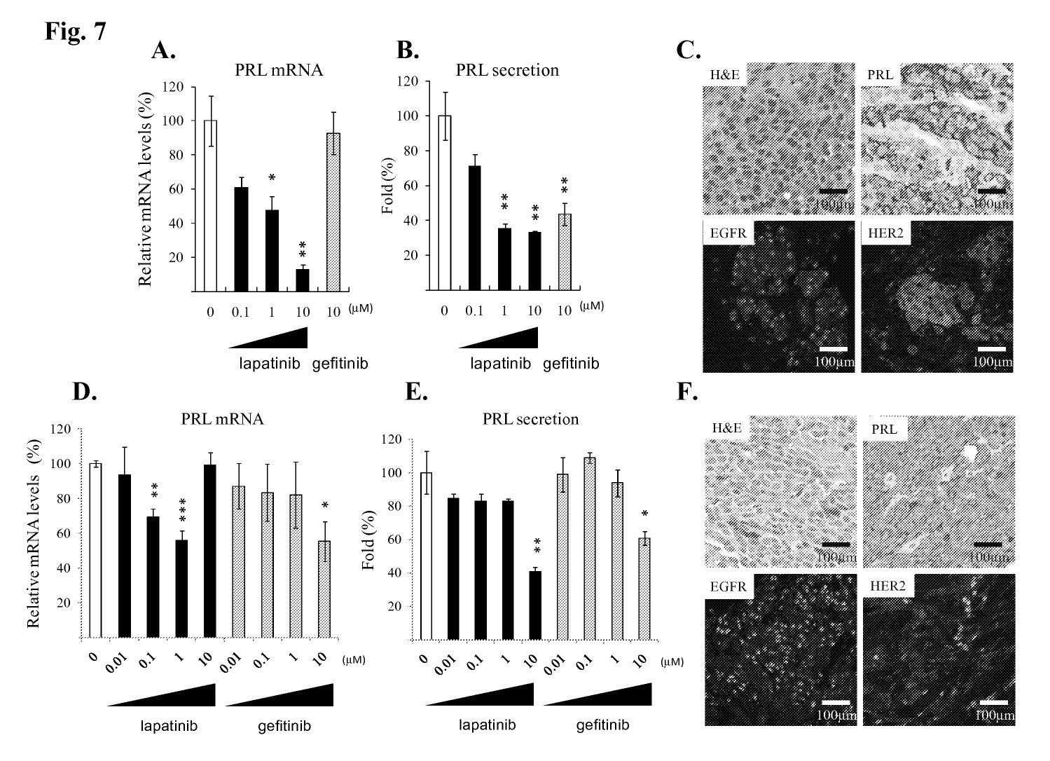

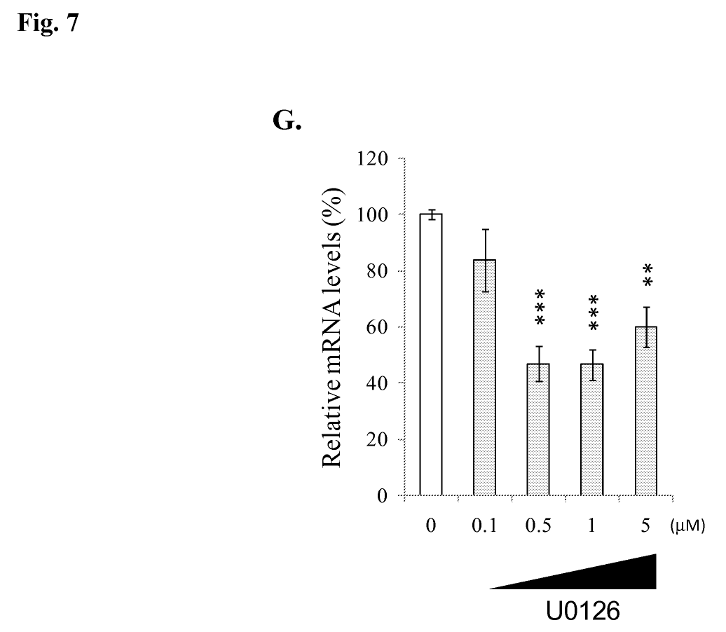

To further support the rationale for clinical use of lapatinib in patients with prolactinoma, The inventors tested drug effects in primary cell cultures derived from two surgically resected prolactinomas. Tumor A prolactinoma cultures showed that PRL mRNA levels as measured by Real-time PCR were markedly suppressed by lapatinib (˜90%, p<0.01), while gefitinib had no effect (FIG. 7A). PRL secretion into the culture medium was also suppressed ˜70% by lapatinib (p<0.01), while gefitinib exhibited more modest PRL suppression (˜50%, FIG. 7B). In cultured prolactinoma cells derived from tumor B, PRL mRNA levels were suppressed by lapatinib (˜45%, p<0.01), and gefitinib by ˜45% (p<0.05, FIG. 7D). PRL secretion into the culture medium was also suppressed ˜60% by lapatinib (p<0.01), while gefitinib exhibited more modest PRL suppression (˜40%, FIG. 7E). As rat PRL induction by EGF is mediated by MAPK (8), The inventor stested U0126, a MEK inhibitor in tumor B cultured cells. In the absence of added EGF, human PRL expression was suppressed ˜55% by U0126 (p<0.001, FIG. 7G).

Using confocal immunofluorescense microscopy, The inventors confirmed expression of both EGFR and HER2 in both prolactinoma tissues, and EGFR appeared localized to cell nuclei. Tumor A expressed HER2 on the membrane and cytoplasm, while in Tumor B, both EGFR and HER2 were detected in nuclei (FIGS. 7C, F). Since both sensitivity and resistance of TKI for ErbB has been associated with an EGFR mutation (25-27). The inventors subjected the patients' pituitary adenoma DNA to sequencing of the EGFR and the HER2 TK domains including exons 18 to 24. No mutation was found in the TK domain exons of either receptors in Tumor A, while two polymorphisms were detected in exon 20 (162093G>A) and exon 23 (179447T>C) of the EGFR in Tumor B. These results suggest that lapatinib suppresses PRL in the absence of such prolactinoma EGFR and HER2 mutations.

### Example 7

The inventors herein show that HER2/ErbB2 overexpression markedly induces PRL gene expression and secretion, and cell growth in rat lactotroph tumor cells. EGF is known to activate PRL transcription (10), and subsequently EGF binding to prolactinoma tissue was reported (28), and EGF and its receptor expression demonstrated in these tumors (29-32). HER2/ErbB2 expression has also been demonstrated in human prolactinomas (9, 19, 33). It is shown herein that prolactinoma HER2/ErbB2 overexpression induces EGFR but not HER3 phosphorylation, with marked increase of PRL, suggesting that PRL induction is mainly mediated by HER2-EGFR heterodimarization rather than HER2-HER3 heterodimerization in these benign hormone-secreting tumors. Interestingly, HER2-HER3 has been shown as the most potently transforming and mitogenic receptor complex of this family in some cancers (34, 35). Mechanisms for PRL induction by EGF have been reported as including MAPK dependent cell pathways (8, 36, 37), and the inventors also reported that HRG, a ligand for HER3, induced PRL in a HER2 and/or HER3 dependent manner (9). In the present study, both PRL mRNA expression and secretion and ERK phosphorylation were more markedly induced by EGF than by HRG in HER2CA cells. Since EGF only binds to EGFR, and HER2/ErbB2 heterodimerizes with all ErbB family members, these results support that HER2-EGFR heterodimers function to induce prolactin.

HER2CA overexpression also induced tumor cell proliferation, at least partially due to MAPK, although the mammalian target of rapamycin (mTOR) pathway has also been implicated in HER2 proliferative actions (13). EGF has been reported to either enhance or attenuate GH3 proliferation (38, 39), and EGF treatment is here shown to enhance colony formation in soft agar. Furthermore, HER2CA inoculated tumor growth was enhanced, suggesting that this receptor overexpression induces prolactinoma cell proliferation.

Since HER2/ErbB2 exhibits such marked selective functions in prolactinoma cells. The inventors focused on this receptor as a therapeutic target for patients with prolactinomas. To determine effects of lapatinib as a novel targeted drug for prolactinoma. The inventors employed four experimental approaches. These included in vitro experiments using HER2CA transfectants, an in vivo allograft model using Wistar-Furth rats inoculated with HER2CA transfectants, another in vivo lactotroph tumor model using Fisher344 rats inoculated with 17β-estradiol, and primary human prolactinoma cell cultures. The inventors used concentrations of lapatinib and gefitinib (0.1 to 10 μM) for in vitro and 100 mg/kg for in vivo experiments as previously shown by others (40-42). Both PRL gene expression and secretion were suppressed by lapatinib in all these experiments. Lapatinib effects were stronger and longer lasting than effects of gefitinib, supporting the critical function of HER2 to induce PRL expression and secretion. Lapatinib also showed superior inhibition of both cell proliferation and tumor growth. Moreover, induction of caspase-3 activity and reduction of soft agar colony formation, together with observed tumor shrinkage in vivo, support an anti-tumorigenic effect of lapatinib in HER2 overexpressing prolactinomas. To assess whether anti-tumorigenic effects of lapatinib are dependent on HER2 expression levels, inventors also treated pcDNA3 transfectants and tested cell number showing that lapatinib suppression of cell number is indeed enhanced in HER2 overexpressing transfectants, suggesting that for prolactinoma, lapatinib is more effective when the tumor overexpresses HER2.

Human prolactinoma HER2 expression was confirmed by immunofluorescence, and nuclear EGFR location was also detected. Nuclear EGFR localization has been reported in breast, ovarian, and thyroid cancers (43-45), likely due to ligand-dependent nuclear translocation of the EGFR (46). Nuclear EGFR may act as a transcription factor (47) and as a direct inducer of PCNA phosphorylation (48). Importantly, nuclear EGFR has been reported to be associated with acquired resistance to cetuximab, a monoclonal antibody directed against the human EGFR ligand binding site (49). However, the results shown herein marked attenuation of PRL gene expression and secretion by lapatinib suggesting that nuclear EGFR does not abrogate lapatinib inhibition of PRL in human prolactinomas expressing HER2. Recently blocking effects of lapatinib were reported on EGFR nuclear translocation (50), consistent with our findings of PRL suppression in the human prolactinoma culture experiments. To assess PRL inhibitory effects of these drugs, RNA levels might be a more accurate indicator, because of more rigorous experimental normalization, suggesting that lapatinib clearly exhibits stronger effects in both tumor A and tumor B. Lower HER2 expression levels, and predominant HER2 nuclear localization could therefore be consistent with less potent suppressive effects of lapatinib in tumor B than in tumor A. Taken together with inventors' previous reports, the HER2 receptor is expressed in 9 of 10 prolactinomas (9). In 8 of 9 tumors, HER2 was located on the membrane suggesting the atypical HER2 nuclear location in tumor B. Furthermore, inventors did not identify an EGFR or HER2 tyrosine kinase domain missense mutation in these tumor cells, yet lapatinib suppressed PRL, suggesting drug efficacy in the absence of such mutations.

The in vitro, in vivo, and human ex vivo results indicate that lapatinib can be a targeted drug as another treatment option for patients with prolactinomas, especially in HER2 overexpressing adenoma. Medical therapies for prolactinomas include the dopamine receptor agonists, bromocriptine and cabergoline (4). Because of its longer lasting and more potent effect, cabergoline is the preferable treatment choice for most patients with prolactinoma (51). Several approaches for treatment of prolactinoma patients resistant to or intolerant of cabergoline include dose increase with rise of concomitant side effects, surgical therapy, radiotherapy, or experimental treatments (4, 52, 53). Temozolomide has been reported as useful for malignant prolactinomas with variable results reported in few cases (52, 54, 55).

The inventors show that HER2/ErbB2 potently induces PRL secretion and regulates experimental prolactinoma cell proliferation. As HER2/ErbB2 pituitary signaling is abrogated by tyrosine kinase inhibitors, especially lapatinib, this receptor could be an effective target for medical therapy of prolactinomas.

### REFERENCES

1. Melmed S 2003 Mechanisms for pituitary tumorigenesis: the plastic pituitary. The Journal of clinical investigation 112:1603-1618
2. Fernandez A, Karavitaki N, Wass J A 2009 Prevalence of pituitary adenomas: a community-based, cross-sectional study in Banbury (Oxfordshire, UK). Clinical endocrinology
3. Ben-Jonathan N, LaPensee C R, LaPensee E W 2008 What can The inventors learn from rodents about prolactin in humans? Endocrine reviews 29:1-41
4. Gillam M P, Molitch M E, Lombardi G, Colao A 2006 Advances in the treatment of prolactinomas. Endocrine reviews 27:485-534
5. Klibanski A 2010 Clinical practice. Prolactinomas. The New England journal of medicine 362:1219-1226
6. Olafsdottir A, Schlechte J 2006 Management of resistant prolactinomas. Nat Clin Pract Endocrinol Metab 2:552-561
7. Casanueva F F, Molitch M E, Schlechte J A, Abs R, Bonert V, Bronstein M D, Brue T, Cappabianca P, Colao A, Fahlbusch R, Fideleff H, Hadani M, Kelly P, Kleinberg D, Laws E, Marek J, Scanlon M, Sobrinho L G, Wass J A, Giustina A 2006 Guidelines of the Pituitary Society for the diagnosis and management of prolactinomas. Clinical endocrinology 65:265-273
8. Vlotides G, Siegel E, Donangelo I, Gutman S, Ren S G, Melmed S 2008 Rat prolactinoma cell growth regulation by epidermal growth factor receptor ligands. Cancer research 68:6377-6386
9. Vlotides G, Cooper O, Chen Y H, Ren S G, Greenman Y, Melmed S 2009 Heregulin regulates prolactinoma gene expression. Cancer research 69:4209-4216
10. Murdoch G H, Potter E, Nicolaisen A K, Evans R M, Rosenfeld M G 1982 Epidermal growth factor rapidly stimulates prolactin gene transcription. Nature 300:192-194
11. Slamon D J, Godolphin W, Jones L A, Holt J A, Wong S G, Keith D E, Levin W J, Stuart S G, Udove J, Ullrich A, et al. 1989 Studies of the HER-2/neu proto-oncogene in human breast and ovarian cancer. Science (New York, N.Y. 244:707-712
12. Yarden Y, Sliwkowski M X 2001 Untangling the ErbB signalling network. Nat Rev Mol Cell Biol 2:127-137
13. Zhang H, Berezov A, Wang Q, Zhang G, Drebin J, Murali R, Greene M I 2007 ErbB receptors: from oncogenes to targeted cancer therapies. The Journal of clinical investigation 117:2051-2058
14. McGuire W L 1987 Prognostic factors for recurrence and survival in human breast cancer. Breast cancer research and treatment 10:5-9
15. Varley J M, Swallow J E, Brammar W J, Whittaker J L, Walker R A 1987 Alterations to either c-erbB-2(neu) or c-myc proto-oncogenes in breast carcinomas correlate with poor short-term prognosis. Oncogene 1:423-430
16. Hirsch F R, Varella-Garcia M, Cappuzzo F 2009 Predictive value of EGFR and HER2 overexpression in advanced non-small-cell lung cancer. Oncogene 28 Suppl 1:S32-37
17. Muller W J, Sinn E, Pattengale P K, Wallace R, Leder P 1988 Single-step induction of mammary adenocarcinoma in transgenic mice bearing the activated c-neu oncogene. Cell 54:105-115
18. Guy C T, Webster M A, Schaller M, Parsons T J, Cardiff R D, Muller W J 1992 Expression of the neu protooncogene in the mammary epithelium of transgenic mice induces metastatic disease. Proceedings of the National Academy of Sciences of the United States of America 89:10578-10582
19. Ezzat S, Zheng L, Smyth H S, Asa S L 1997 The c-erbB-2/neu proto-oncogene in human pituitary tumours. Clinical endocrinology 46:599-606
20. Roncaroli F, Nose V, Scheithauer B W, Kovacs K, Horvath E, Young W F, Jr., Lloyd R V, Bishop M C, Hsi B, Fletcher J A 2003 Gonadotropic pituitary carcinoma: HER-2/neu expression and gene amplification. Report of two cases. Journal of neurosurgery 99:402-408
21. Nose-Alberti V, Mesquita M I, Martin L C, Kayath M J 1998 Adrenocorticotropin-Producing Pituitary Carcinoma with Expression of c-erbB-2 and High PCNA Index: A Comparative Study with Pituitary Adenomas and Normal Pituitary Tissues. Endocrine pathology 9:53-62
22. Rusnak D W, Lackey K, Affleck K, Wood E R, Alligood K J, Rhodes N, Keith B R, Murray D M, Knight W B, Mullin R J, Gilmer T M 2001 The effects of the novel, reversible epidermal growth factor receptor/ErbB-2 tyrosine kinase inhibitor, GW2016, on the growth of human normal and tumor-derived cell lines in vitro and in vivo. Molecular cancer therapeutics 1:85-94
23. Mok T S, Wu Y L, Thongprasert S, Yang C H, Chu D T, Saijo N, Sunpaweravong P, Han B, Margono B, Ichinose Y, Nishiwaki Y, Ohe Y, Yang J J, Chewaskulyong B, Jiang H, Duffield E L, Watkins C L, Armour A A, Fukuoka M 2009 Gefitinib or carboplatin-paclitaxel in pulmonary adenocarcinoma. The New England journal of medicine 361:947-957
24. Heaney A P, Horwitz G A, Wang Z, Singson R, Melmed S 1999 Early involvement of estrogen-induced pituitary tumor transforming gene and fibroblast growth factor expression in prolactinoma pathogenesis. Nat Med 5:1317-1321
25. Wang Q, Greene M I 2008 Mechanisms of resistance to ErbB-targeted cancer therapeutics. The Journal of clinical investigation 118:2389-2392
26. Rosell R, Moran T, Queralt C, Porta R, Cardenal F, Camps C, Majem M, Lopez-Vivanco G, Isla D, Provencio M, Insa A, Massuti B, Gonzalez-Larriba J L, Paz-Ares L, Boyer I, Garcia-Campelo R, Moreno M A, Catot S, Rolfo C, Reguart N, Palmero R, Sanchez J M, Bastus R, Mayo C, Bertran-Alamillo J, Molina M A, Sanchez J J, Taron M 2009 Screening for epidermal growth factor receptor mutations in lung cancer. The New England journal of medicine 361:958-967
27. Cappuzzo F, Varella-Garcia M, Shigematsu H, Domenichini I, Bartolini S, Ceresoli G L, Rossi E, Ludovini V, Gregorc V, Toschi L, Franklin W A, Crino L, Gazdar A F, Bunn P A, Jr., Hirsch F R 2005 Increased HER2 gene copy number is associated with response to gefitinib therapy in epidermal growth factor receptor-positive non-small-cell lung cancer patients. J Clin Oncol 23:5007-5018
28. Jaffrain-Rea M L, Petrangeli E, Lubrano C, Minniti G, Di Stefano D, Sciarra F, Frati L, Tamburrano G, Cantore G, Gulino A 1998 Epidermal growth factor binding sites in human pituitary macroadenomas. The Journal of endocrinology 158:425-433
29. Kontogeorgos G, Stefaneanu L, Kovacs K, Cheng Z 1996 Localization of Epidermal Growth Factor (EGF) and Epidermal Growth Factor Receptor (EGFr) in Human Pituitary Adenomas and Nontumorous Pituitaries: An Immunocytochemical Study. Endocrine pathology 7:63-70
30. LeRiche V K, Asa S L, Ezzat S 1996 Epidermal growth factor and its receptor (EGF-R) in human pituitary adenomas: EGF-R correlates with tumor aggressiveness. The Journal of clinical endocrinology and metabolism 81:656-662
31. Theodoropoulou M, Arzberger T, Gruebler Y, Jaffrain-Rea M L, Schlegel J, Schaaf L, Petrangeli E, Losa M, Stalla G K, Pagotto U 2004 Expression of epidermal growth factor receptor in neoplastic pituitary cells: evidence for a role in corticotropinoma cells. The Journal of endocrinology 183:385-394
32. Onguru O, Scheithauer B W, Kovacs K, Vidal S, Jin L, Zhang S, Ruebel K H, Lloyd R V 2004 Analysis of epidermal growth factor receptor and activated epidermal growth factor receptor expression in pituitary adenomas and carcinomas. Mod Pathol 17:772-780
33. Chaidarun S S, Eggo M C, Sheppard M C, Stewart P M 1994 Expression of epidermal growth factor (EGF), its receptor, and related oncoprotein (erbB-2) in human pituitary tumors and response to EGF in vitro. Endocrinology 135:2012-2021
34. Tzahar E, Waterman H, Chen X, Levkowitz G, Karunagaran D, Lavi S, Ratzkin B J, Yarden Y 1996 A hierarchical network of interreceptor interactions determines signal transduction by Neu differentiation factor/neuregulin and epidermal growth factor. Molecular and cellular biology 16:5276-5287
35. Pinkas-Kramarski R, Soussan L, Waterman H, Levkowitz G, Alroy I, Klapper L, Lavi S, Seger R, Ratzkin B J, Sela M, Yarden Y 1996 Diversification of Neu differentiation factor and epidermal growth factor signaling by combinatorial receptor interactions. The EMBO journal 15:2452-2467
36. Ben-Jonathan N, Chen S, Dunckley J A, LaPensee C, Kansra S 2009 Estrogen receptor-alpha mediates the epidermal growth factor-stimulated prolactin expression and release in lactotrophs. Endocrinology 150:795-802
37. Chen S, Bangaru M L, Sneade L, Dunckley J A, Ben-Jonathan N, Kansra S 2009 Epidermal growth factor receptor cross-talks with ligand-occupied estrogen receptor-alpha to modulate both lactotroph proliferation and prolactin gene expression. American journal of physiology 297:E331-339
38. Johnson L K, Vlodaysky I, Baxter J D, Gospodarowicz D 1980 Nuclear accumulation of epidermal growth factor in cultured rat pituitary cells. Nature 287:340-343
39. Schonbrunn A, Krasnoff M, Westendorf J M, Tashjian A H, Jr. 1980 Epidermal growth factor and thyrotropin-releasing hormone act similarly on a clonal pituitary cell strain. Modulation of hormone production and inhbition of cell proliferation. The Journal of cell biology 85:786-797
40. Nahta R, Yuan L X, Du Y, Esteva F J 2007 Lapatinib induces apoptosis in trastuzumab-resistant breast cancer cells: effects on insulin-like growth factor I signaling. Mol Cancer Ther 6:667-674
41. Gril B, Palmieri D, Bronder J L, Herring J M, Vega-Valle E, Feigenbaum L, Liewehr D J, Steinberg S M, Merino M J, Rubin S D, Steeg P S 2008 Effect of lapatinib on the outgrowth of metastatic breast cancer cells to the brain. J Natl Cancer Inst 100:1092-1103
42. Chu I, Blackwell K, Chen S, Slingerland J 2005 The dual ErbBl/ErbB2 inhibitor, lapatinib (GW572016), cooperates with tamoxifen to inhibit both cell proliferation- and estrogen-dependent gene expression in antiestrogen-resistant breast cancer. Cancer Res 65:18-25
43. Lo H W, Xia W, Wei Y, Ali-Seyed M, Huang S F, Hung M C 2005 Novel prognostic value of nuclear epidermal growth factor receptor in breast cancer. Cancer research 65:338-348
44. Marti U, Ruchti C, Kampf J, Thomas G A, Williams E D, Peter H J, Gerber H, Burgi U 2001 Nuclear localization of epidermal growth factor and epidermal growth factor receptors in human thyroid tissues. Thyroid 11:137-145
45. Xia W, Wei Y, Du Y, Liu J, Chang B, Yu Y L, Huo L F, Miller S, Hung M C 2009 Nuclear expression of epidermal growth factor receptor is a novel prognostic value in patients with ovarian cancer. Mol Carcinog 48:610-617
46. Lo H W, Hsu S C, Ali-Seyed M, Gunduz M, Xia W, Wei Y, Bartholomeusz G, Shih J Y, Hung M C 2005 Nuclear interaction of EGFR and STAT3 in the activation of the iNOS/NO pathway. Cancer cell 7:575-589
47. Lin S Y, Makino K, Xia W, Matin A, Wen Y, Kwong K Y, Bourguignon L, Hung M C 2001 Nuclear localization of EGF receptor and its potential new role as a transcription factor. Nature cell biology 3:802-808
48. Wang S C, Nakajima Y, Yu Y L, Xia W, Chen C T, Yang C C, Mclntush E W, Li L Y, Hawke D H, Kobayashi R, Hung M C 2006 Tyrosine phosphorylation controls PCNA function through protein stability. Nature cell biology 8:1359-1368
49. Li C, Iida M, Dunn E F, Ghia A J, Wheeler D L 2009 Nuclear EGFR contributes to acquired resistance to cetuximab. Oncogene 28:3801-3813
50. Kim H P, Yoon Y K, Kim J W, Han S W, Hur H S, Park J, Lee J H, Oh D Y, Im S A, Bang Y J, Kim T Y 2009 Lapatinib, a dual EGFR and HER2 tyrosine kinase inhibitor, downregulates thymidylate synthase by inhibiting the nuclear translocation of EGFR and HER2. PLoS One 4:e5933
51. Schlechte J A 2003 Clinical practice. Prolactinoma. The New England journal of medicine 349:2035-2041
52. Goffin V, Bernichtein S, Touraine P, Kelly P A 2005 Development and potential clinical uses of human prolactin receptor antagonists. Endocrine reviews 26:400-422
53. Passos V Q, Fortes M A, Giannella-Neto D, Bronstein M D 2009 Genes differentially expressed in prolactinomas responsive and resistant to dopamine agonists. Neuroendocrinology 89:163-170
54. Kovacs K, Horvath E, Syro L V, Uribe H, Penagos L C, Ortiz L D, Fadul C E 2007 Temozolomide therapy in a man with an aggressive prolactin-secreting pituitary neoplasm: Morphological findings. Human pathology 38:185-189
55. McCormack A I, McDonald K L, Gill A J, Clark S J, Burt M G, Campbell K A, Braund W J, Little N S, Cook R J, Grossman A B, Robinson B G, Clifton-Bligh R J 2009 Low O6-methylguanine-DNA methyltransferase (MGMT) expression and response to temozolomide in aggressive pituitary tumours. Clinical endocrinology 71:226-233
56. Stewart C F, Leggas M, Schuetz J D, Panetta J C, Cheshire P J, Peterson J, Daw N, Jenkins III J J, Gilbertson R, Germain G S, Harwood F C, Houghton P J 2004 Gefitinib enhances the antitumor activity and oral bioavailability of irinotecan in mice. Cancer Res 64:7491-7499
57. Leggas M, Panetta J C, Zhuang Y, Schuetz J D, Johnston B, Bai F, Sorrentino B, Zhou S, Houghton P J, Stewart C F 2006 Gefitinib modulates the function of mulitple ATP-binding cassette transporters in vivo. Cancer Res 66:4802-4807
58. Ueda S, Basaki Y, Yoshie M, Ogawa K, Sakisaka S, Kuwano M, Ono M 2006 PTEN/Akt signaling through epidermal growth factor receptor is prerequisite for angiogenesis by hepatocellular carcinoma cells that is susceptible to inhibition by gefitinib. Cancer Res 66:5346-5353
59. Fabian M A, Biggs III W H, Treiber D K, Atteridge C E, Azimioara M D, Benedetti M G, Carter T A, Ciceri P, Edeen P T, Floyd M, Ford J M, Galvin M, Gerlach J L, Grotzfeld R M, Herrgard S, Insko D E, Insko M A, Lai A G, Lelias J M, Mehta S A, Milanov Z V, Velasco A M, Wodicka L M, Patel H K, Zarrinkar P P, et al. 2005 A small molecule-kinase interaction map for clinical kinase inhibitors. Nat Biotechnol 23:329-336
60. Sirotnak F M, Zakowski M F, Miller V A, Scher H I, Kris M G 2000 Efficacy of cytotoxic agents against human tumor xenografts is markedly enhanced by coadministration of ZD1839 (Iressa), an inhibitor of EGFR tyrosine kinase. Clin Cancer Res 6:4885-4892

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method for treating prolactinoma in a subject in need thereof, comprising:
(i) providing a composition comprising a tyrosine kinase inhibitor; and
(ii) administering a therapeutically effective amount of the composition to the subject to treat the prolactinoma, thereby treating prolactinoma in the subject.

2. A method for inhibiting prolactinoma in a subject in need thereof, comprising:
(i) providing a composition comprising a tyrosine kinase inhibitor; and
(ii) administering a therapeutically effective amount of the composition to the subject to inhibit the prolactinoma, thereby inhibiting prolactinoma in the subject.

3. A method for reducing prolactinoma tumor size in a subject in need thereof comprising:
(i) providing a composition comprising a tyrosine kinase inhibitor; and
(ii) administering a therapeutically effective amount of the composition to the subject to reduce the prolactinoma, thereby reducing the prolactinoma tumor size in the subject.

4. A method for promoting prolactinoma prophylaxis in subject in need thereof comprising:
(i) providing a composition comprising a tyrosine kinase inhibitor; and
(ii) administering a therapeutically effective amount of the composition to the subject to promote prolactinoma prophylaxis thereby promoting prolactinoma prophylaxis in the subject.

5. The method of claim 1, wherein the tyrosine kinase inhibitor is selected from the group consisting of a small molecule, a peptide, an antibody or a fragment thereof and a nucleic acid molecule.

6. The method of claim 4, wherein the tyrosine kinase inhibitor is lapatinib.

7. The method of claim 4, wherein the tyrosine kinase inhibitor is gefitinib.

8. The method of claim 4, wherein the nucleic acid molecule is an siRNA molecule of tyrosine kinase.

9. The method of claim 4, wherein the antibody is selected from the group consisiting of monoclonal antibody or fragment thereof, a polyclonal antibody or a fragment thereof, chimeric antibodies, humanized antibodies, human antibodies, and a single chain antibody.

10. The method of claim 1, wherein the tyrosine kinase inhibitor is administered intravenously, intramuscularly, intraperitonealy, orally or via inhalation.

11. A method of claim 1, wherein the effective amount of the tyrosine kinase inhibitor is about 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day or 1900-2000 mg/day.

12. A method for identifying inhibitors of tyrosine kinase comprising:
(i) contacting the tyrosine kinase in tyrosine kinase positive cells with a molecule of interest, and
(ii) determining whether the contact results in decreased secretion of prolactin, a decrease in prolactin secretion being indicative that the molecule of interest is an inhibitor of tyrosine kinase.

13. The method of claim 12, wherein the tyrosine kinase inhibitor is selected from the group consisting of a small molecule, a peptide, an antibody or a fragment thereof and a nucleic acid molecule.

14. A screening method according to claim 12, which comprises separately contacting each of a plurality of samples to be tested.

15. The screening method of claim 14, wherein the plurality of samples comprises more than about 10⁴ samples.

16. The screening method of claim 14, wherein the plurality of samples comprises more than about 5×10⁴ samples.

17. The method of claim 1, wherein the subject is selected from the group consisting of human, non-human primate, monkey, ape, dog, cat, cow, horse, rabbit, mouse and rat.

18. A kit for the treatment of prolactinoma, inhibition of prolcatinoma, reduction of prolactinoma or promotion of prolactinoma prophylaxis in a subject in need thereof, comprising:
(i) a composition comprising a tyrosine kinase inhibitor; and
(ii) instructions for use of the composition for the treatment of prolactinoma, inhibition of prolactinoma, reduction of prolactinoma or promotion of prolactinoma prophylaxis.

19. The method of claim 17, wherein the tyrosine kinase inhibitor is lapatanib.
